(19) United States
(12) Patent Application Publication
(10) Pub. No.: US 2006/0184190 A1
(43) Pub. Date: Aug. 17, 2006

(54) Trans-myocardial fluid-jet revascularization arrangement

(76) Inventors: Ernest M. Feiler, Lafayette, CO (US); Alex Wormser, Marblehead, MA (US); Frederic C. Feiler, Raleigh, NC (US); Warren Roh, Littleton, CO (US)

Correspondence Address: Donald N. Halgren, 35 Central Street, Manchester, MA, 01944, US

(21) Appl. No.: 11/057,613

(22) Filed: Feb. 14, 2005

(51) Int. Cl. A61B 17/34

(52) U.S. Cl. 606/185

(57) ABSTRACT

A surgical procedure for revascularization the myocardium, comprising the steps of: directing a generally cylindrically shaped first nozzle into a heart wall being treated; forming a first or main channel in the heart wall from the ventricle into the myocardium of the heart by the nozzle; removing a generally cylindrically shaped tissue core through the nozzle, from the heart wall during formation of the first or main channel. A set of tributary channels may be made in the heart wall from and in communication with the main channel by an arrangement of radially directed fluid jets subsequent to the creation of that main channel.

### BACKGROUND OF THE INVENTION

1. Field of the Invention

The present invention relates to arrangements for bringing blood flow into the myocardium through channels that are connected directly to the ventricle.

2. Prior Art

Coronary bypass surgery consists of bringing blood from a source of pressure through grafts that are attached to the coronary arteries where they have been surgically opened beyond the obstructed area. If the coronary arteries were too small or too severely diseased for such surgery, they were also too small for balloon dilation and stent insertion. Therefore, these patients were left with incomplete revascularizations. This could lead to subsequent need for re-operation for angina, heart attacks, rhythm disturbances or death.

Coronary arteriograms show only the larger arteries, and can not show the small arteries. Therefore, incomplete revascularization may not be recognized when it is due to branch occlusions, such as in diabetics, old people, and patients after a heart attack.

In areas of inadequate perfusion as above, mechanical methods were attempted to make ventriculo-myocardial channels using primarily cannulas or trocars. They failed apparently because they produced slits instead of holes.

A system using lasers to create ventriculo-myocardial channels was used more successfully and became the recognized alternative surgical approach when coronary bypass grafting and angioplasty was not feasible. Unfortunately the laser channels often closed as well. The closure of these channels is postulated to be due to the high temperature generated by the laser which causes scar formation around the channels that in turn close them.

It is the object of this invention to revascularize hearts that are not amenable to coronary bypass graft surgery or angioplasty, thereby increasing the life expectancy and quality of life of these patients. This is accomplished by the formation of ventriculo-myocardial channels that remain open, by creating tributary channels that increase the distribution of blood through the myocardium, and by creating a system that can be readily accepted, both in ease of use and in cost.

### BRIEF SUMMARY OF THE INVENTION

In a preferred embodiment of the invention, a series of main channels are formed by a nozzle that emits jets of high pressure fluid from an annular orifice placed on the heart's surface. The nozzle and high pressure fluid are advanced through the myocardium. This proces is called TMFR or Trans Myocardial Fluid-Jet Revascularization.

Tributary channels may also be formed from each main channel by high pressure jets emitted from radial orifices in the surface of a sealed chamber surrounding the nozzle.

The fluid-Jets may create debris during formation of these main channels and tributaries in the heart's wall. This debris must be kept out of the bloodstream, to prevent embolization. In the formation of the main channels, except at the instant of penetration, the spent cutting fluid and any cutting debris it contains is kept isolated from the bloodstream by the uncut section of the myocardium. Debris formed at the moment of penetration is suctioned away through a tube at the center of the nozzle and is disposed of. Similarly, fluid used to form the tributary channels is kept isolated from the blood stream.

The system for supplying the fluid-Jet with fluid includes a reservoir containing fluid, a pressure pump, and a control system including solenoid valves for starting and stopping the fluid flows. Typically, a gear pump is used to generate the pressure in the fluid supplied to the nozzle arrangement. The fluid-Jet fluid is a physiologic solution such a Lactated Ringer's Solution. A signal from an electrocardiogram may be connected to a control unit computer and is used to synchronize the start of flow of each pressurized stream with the pause between heartbeats.

The thickness of the heart wall as measured by ultrasound is used to controllably adjust a mechanical stop in a collector ring adjacent the distal end of the nozzle to correctly limit the nozzle's depth of penetration through the myocardium. This permits the nozzle to penetrate far enough to form the channel, but not so far that the seal at the distal end of the nozzle loses contact with the newly-formed channel. Cuts created by Fluid-Jets extend beyond their orifices, so the adjustment of the stop on the nozzle is an approximation only.

Procedurally, the system's control system is then turned on, including the pressure and suction pumps. At the start of the revascularization, the annular opening of the collector ring disposed circumferentially on the nozzle is centered over the locus of the new main channel. The probe is inserted through the opening, and the fluid-Jet fluid flow is started. As the main channel is formed, the probe is pushed into the wall of the heart until is restrained by the stop on the collector ring touching the heart wall. The fluid is immediately turned off to minimize the cutting debris from mixing with the blood stream and also to minimize damage to the formed elements for blood flow.

Penetration of the heart wall causes a sudden loss of vacuum in the nozzle's inner suction tube, and its measurement (loss of vacuum), may be used to automatically shut off the tissue-penetrating fluid-Jet flow. The drop in pressure also confirms that the penetration is complete. Alternatively, the probe's contact of a switch in the collector ring's mechanical stop with the heart's surface may be used to shut off the fluid-Jet fluid flow.

A further step in the procedure occurs when the pressurized fluid to the tributary nozzle orifices is turned on for a predetermined period. The fluid pressure is then reduced below that required for cutting for an additional period, to permit the flushing away of any remaining debris. The fluid-Jet flow is then turned off, and the probe and collector ring are removed. The entrance to the main channel on the heart surface is sutured if required.

The procedure is then repeated as needed. Typically, five to eight channels are required per square inch to treat poorly perfused myocardium, less then the number needed with TMLR. Ideally, channels are created on alternate sides of diseased arteries at a spacing of about 1-1½ inches.

The fluid pressure at the orifice must be at least 1000 to 2000 psi to cut the tissue. Higher pressures reduce the cutting time, but the system's maximum pressure is limited by the strength of the nozzle tube and the stiffness of the hoses. The nozzle's outer diameter is determined by the space requirements of its tubing and flow passages. The diameter of the main channel generated in a heart wall is determined by the diameter of the annular orifice, which is smaller than the nozzle's outer diameter. A typical main channel diameter is expected to average about 0.04″, while a typical nozzle's outer diameter may be about 0.07″ if there are tributary fluid-jets, and a diameter of about 0.05″ in the single probe nozzle embodiment (without the tributary forming side nozzles). The stretchiness of the myocardium is expected to allow the heart's wall to accommodate the diametrical interference without difficulty.

The tributary channels generated by the side nozzles in the nozzle probe apparatus are expected to range from about ⅜ to ¾″ in length and about 0.02 to 0.04″ in diameter. Typically, there are preferably about six tributary side orifices per probe, positioned 180 degrees apart around the circumference of the nozzle and fairly evenly distributed through the thickness of the myocardium. When used near the inter-ventricular septum, no tributary channels may extend toward the septum, to avoid injury to the conducting bundles. Generally, the direction of the tributaries is roughly perpendicular to the main channel and parallel to the surface of the heart.

Tributary channels may be created with heating the fluid-jet fluid to attempt to cause angioneogenesis, but not heated so hot so as to cause denaturing of protein and scarring of the heart wall.

An alternative embodiment of the present invention is to form the main channels in the heart wall by the utilization of a manually pressed sharpened cannula instead against the wall of the heart instead of a pressurized fluid-Jet to cut the core. The cannula would have distal end with a sharpened edge oriented externally, and removal of debris would be accomplished by suction through a central lumen of the cannula. Residual core material is removed after each penetration and subsequent withdrawal, preferably by a pulse of high pressure fluid introduced at the cannula's proximal end. The cannula may be in yet a further embodiment, be fitted with an outer fluid-jet jacket with appropriate radially directed nozzles therein, to create tributary channels and to provide angioneogenesis in a manner similar to the aforementioned fluid-jet nozzle embodiments.

The invention thus comprises a surgical procedure for revascularization the myocardium, comprising one or more of the following steps: directing a generally cylindrically shaped first nozzle into a heart wall being treated; forming a first or main channel in the heart wall from the ventricle into the myocardium of the heart by the nozzle; removing a generally cylindrically shaped tissue core through the nozzle, from the heart wall during formation of the first or main channel; placing a heart wall tissue cutting arrangement on a distal end of the nozzle; and placing a heart wall tissue withdrawing arrangement on a proximal end of the nozzle. The surgical procedure may include: supporting a first conduit co-axially around the first nozzle so as to form a longitudinally directed first annular passageway between the nozzle and the first conduit; providing a pressurized cutting fluid into the first passageway from a controllable pressurized fluid source to direct pressurized fluid from a distal end of the nozzle, for cutting the heart wall. The surgical procedure may include one or more of the following: a sharpened annular edge on the distal end of the nozzle. The surgical procedure may comprise a vacuum conduit in communication with the first nozzle to facilitate removal of the tissue core from the heart wall and through the first nozzle; forming a chamfered distal end on the nozzle and the first conduit so as to direct the cutting fluid in an inward conically shaped direction to reduce the diameter of a tissue core removed from the wall; arranging a plurality of sideway directed orifices through the first conduit adjacent the distal end thereof, and jetting a pressurized fluid through the sideway directed orifices to generate a plurality of tributary channels in the main channel in said wall of the heart being treated; inserting a second conduit between the nozzle and the first conduit to define a further annular passageway; introducing a pressurized fluid into both the first annular passageway and the second passageway from a pressurized fluid source, to provide tissue cutting of the heart wall and to provide tissue debris removal means therewith; arranging a tissue engaging nose piece arrangement on the distal end of the second conduit so as to provide a tissue sealing arrangement adjacent an annular pressurized fluid-emitting orifice thereat; moving the nozzle longitudinally so as to dimensionally alter the annular pressurized fluid emitting orifice; placing a longitudinally adjustable heart wall engaging plenum adjacent the distal end of the nozzle; attaching the plenum into communication with a vacuum source to remove debris from the heart wall during a channel generating revascularization procedure; forming a main channel into a heart wall; forming a plurality of tributaries generally perpendicular with respect to the main channel and into the wall of the heart; and evacuating debris from the tributaries through an outer annular channel while evacuating core tissue from the main channel through the nozzle.

The invention may also comprise an apparatus for performing a revascularization procedure on a myocardium, comprising: an elongated hollow nozzle having a proximal end and a tissue piercing distal end; and a first conduit arranged co-axial with the nozzle to define an annular fluid directing passageway therebetween, and a controllable pressure source in communication with the passageway.

The apparatus may include a vacuum arranged in communication with the proximal end of the nozzle to withdraw a core plug of tissue from the nozzle; an annular plenum for sealing the nozzle during a procedure and for withdrawing debris generated therewith. The plenum may have an adjustment means thereon to limit the depth of a nozzle may travel into a heart wall.

### BRIEF DESCRIPTION OF THE DRAWINGS

The objects and advantages of the present invention will become more apparent when viewed in conjunction with the following drawings, in which:

FIG. 1 is a side elevational view of a fluid-emitting tissue-piercing probe assembly constructed according to the principles of the present invention;

FIG. 2a is a side elevational view, in longitudinal section, of a nozzle for forming a main channel in a heart wall;

FIG. 2b is a view similar to FIG. 2a showing a nozzle forming a main channel in a heart wall, removing a longitudinal segment thereof;

FIG. 3 is a side elevational view, in longitudinal section, of a nozzle arrangement, including an outer conduit arranged for forming tributaries in a heart wall;

FIG. 4 is a side elevational view of the nozzle arrangement shown in FIG. 3, with a collector ring arranged thereon for removal of debris from the channel formation procedure;

FIG. 5 is a schematic representation of a pressurized fluid source which is arranged to provide pressurized fluid to the nozzle assembly of the present invention;

FIG. 6 is a side elevational view in longitudinal section of a cannula for forming a main channel in a heart wall; and

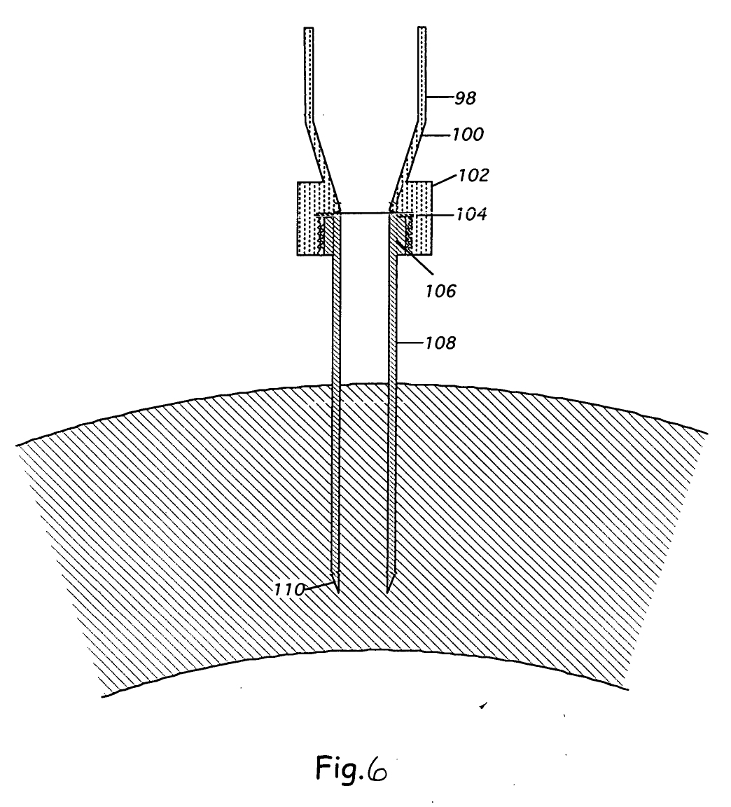

FIG. 7 is a further embodiment of the nozzle shown in FIG. 6 using a cannula and an outer conduit for forming a main channel and tributary channels in a heart wall.

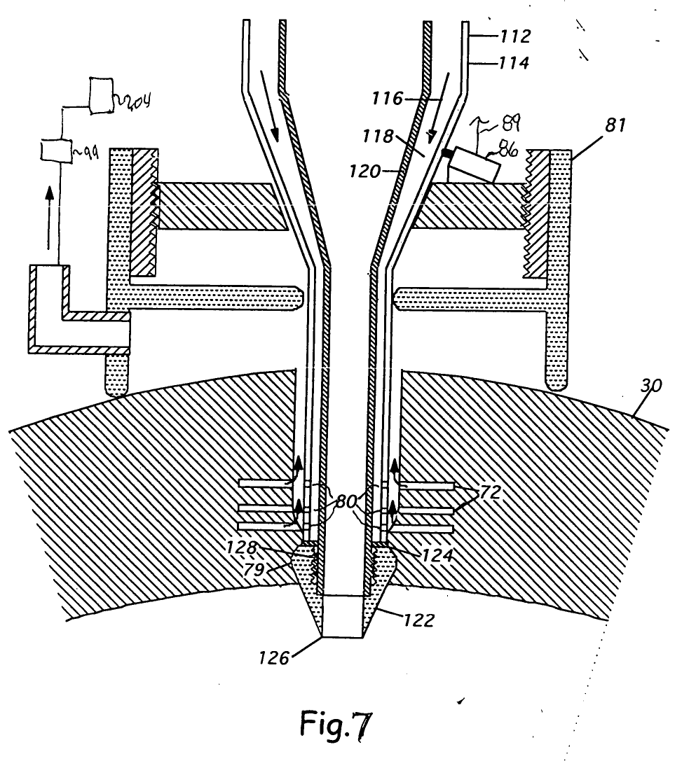

### DETAILED DESCRIPTION OF THE PREFERRED EMBODIMENTS

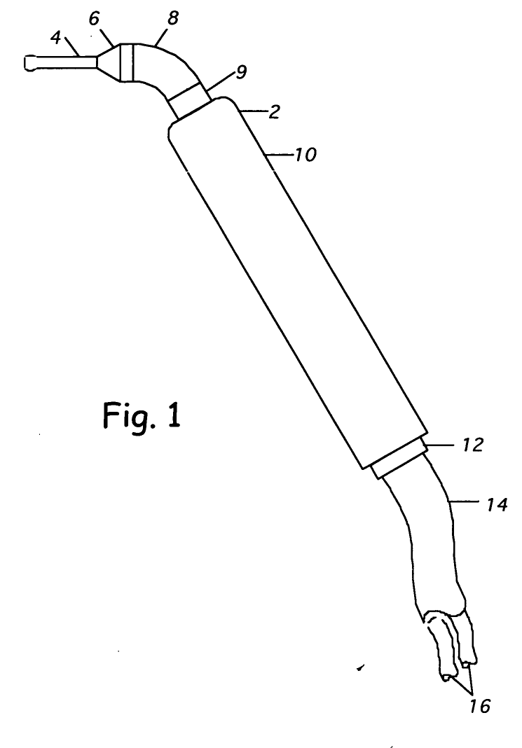

Referring now to the drawings in detail, and particularly to FIG. 1, there is shown the present invention which includes a nozzle assembly or probe 2 for performing trans-myocardial fluid-jet revascularization. The probe 2 comprises an elongated, heart wall tissue withdrawing nozzle 4 which is attached to and in fluid communication with an expander 6, an elbow 8, and a securement conduit 9. The diameter of nozzle 4 is small because the main channels to be formed in a heart wall are small. The diameter of conduit 9 is larger than the nozzle 4, for greater strength and also to reduce drops of fluid flow pressure therethrough. The expander 6 is arranged to connect the nozzle 4 to the elbow 8.

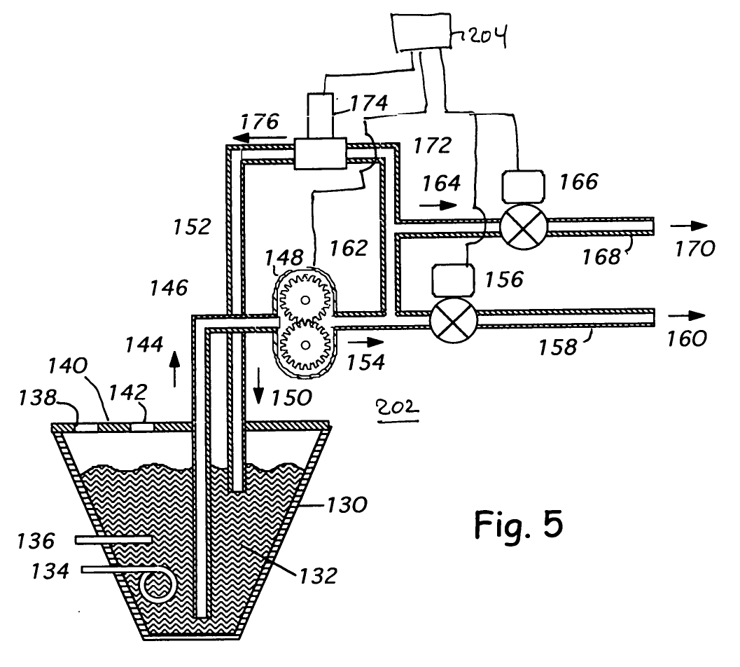

An elongated hollow, fluid conduit enclosing handle 10 is communicatively attached to the securement conduit 9. The securement conduit 9 and internal conduits from the nozzle, if any, are attached in a leak-proof manner to a proximal connector 12. Openings in the proximal connector 12 are connected in a leak-proof manner to one or more pressure or tissue carrying hoses 16 contained in an enclosure cable 14. The enclosure cable 14 is also attached to the proximal end of the proximal connector 12. One embodiment of the invention includes the enclosure cable 14 carrying an internal hose or cable for transmitting a pressure signal to a control system 11, disclosed in FIG. 5.

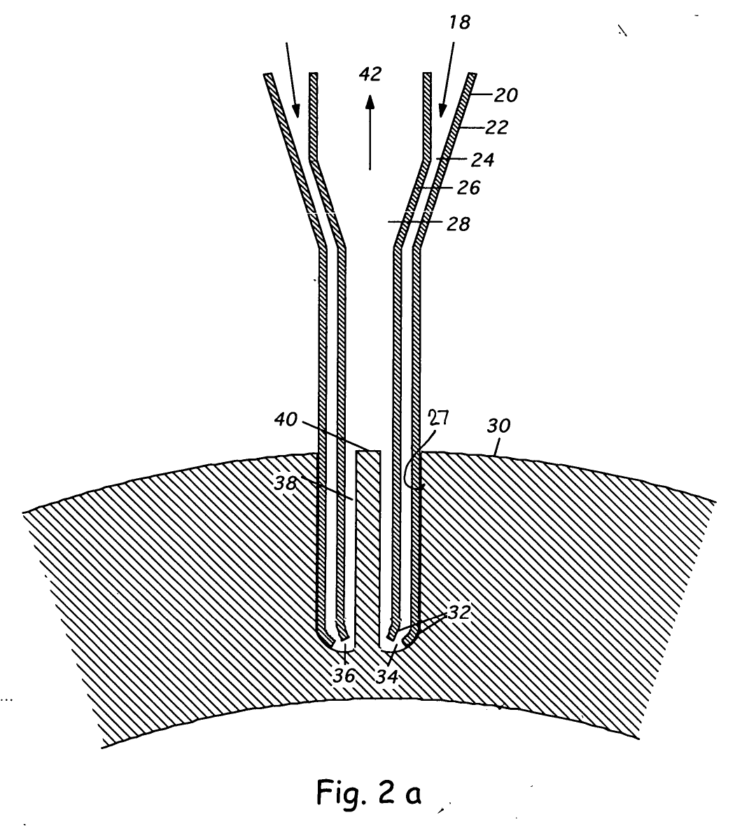

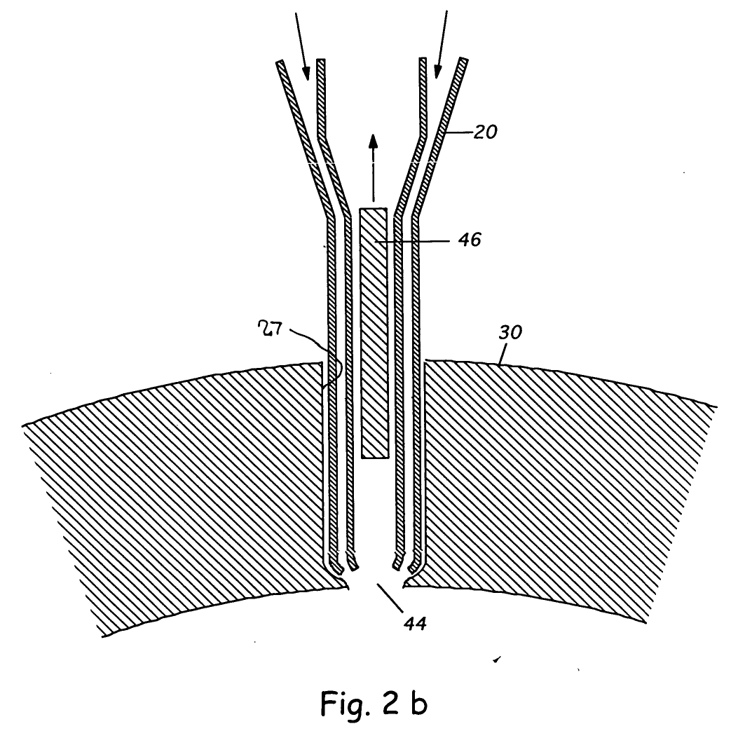

The probe or nozzle assembly in one preferred embodiment includes a nozzle 20 as shown in FIG. 2a which nozzle 20 is used for forming a main channel arrangement 27 in a heart wall 30. The FIG. 2a shows the nozzle 20 as it appears before the completion of the main channel 27 through the heart wall 30. The nozzle assembly comprises an outer conduit 22 that forms the outer boundary of an annular passage 24 for conveying pressurized fluid 18 from a source 130, as shown in FIG. 5, to an annular orifice 34 in the distalmost end of the nozzle 20. As with all of the nozzles of the present invention, the proximal end of the tubular conduit that penetrates the heart's wall 30 is preferably tapered outwardly, so as to be attached to the conically shaped expander 6, which is shown in FIG. 1.

The inner boundary of channel 24 is defined by an inner conduit 26. The distalmost ends 32 of both conduits 22 and 26 are chambered radially inwardly to deflect the annular fluid-jet flow inwardly. This causes the outer diameter of a heart wall core 38 being removed to be smaller than the inside diameter of the inner conduit 26, preventing the heart wall core 38 from becoming stuck during removal attempts by suction, through the inner conduit 26.

The fluid-jet forms the main channel by removing heart tissue and creating an annular space 36. The spent fluid is emitted through an innermost channel 40 within inner conduit 26, and out through the vacuum system at a distal outlet 42 in the conduit 26.

The same nozzle 20 is shown in FIG. 2b, as it appears just after the main channel 27 has been extended so as to penetrate the heart wall 30, forming an inner opening 44. The penetration of the nozzle 20 has caused the wall core material 46 to become detached from the heart wall 30, allowing it to be suctioned away through inner conduit 26.

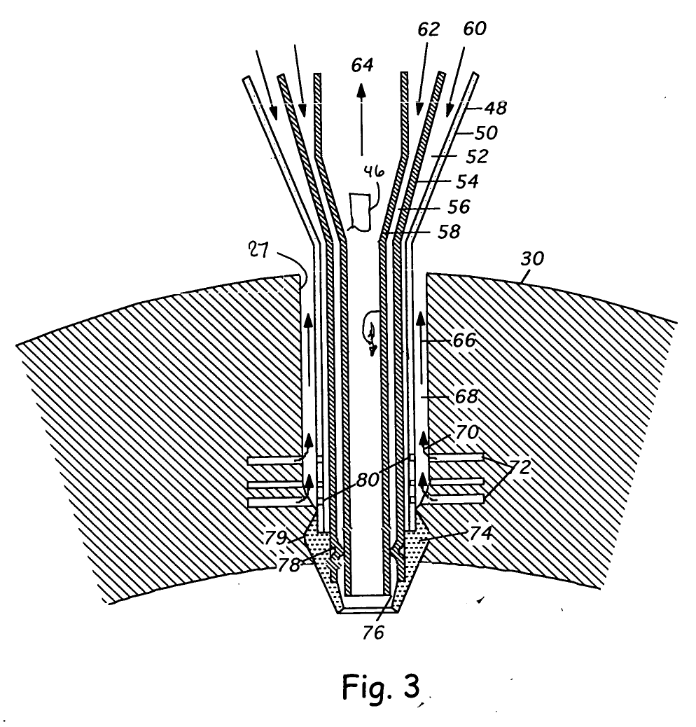

An embodiment of a combination nozzle 48 is shown in FIG. 3, which nozzle 48 may be utilized to form both the main channel 27 and a plurality of tributary channels 72. The nozzle 48 provides an outer passage 52 for fluid-jet fluid 60 to produce the tributary channels 72 from radially directed orifices 80 in an outer conduit 50. The outer passage 52 is bounded by the outer conduit 50 and a middle conduit 54. The nozzle 48 also includes an inner passage 56 for conveying main channel fluid-jet fluid 62 from its pressure source shown in FIG. 5, to an annular orifice 76 defined by the annular gap at the distalmost ends of the middle and innermost conduits 54 and 58 and the interior of an annularly disposed, tissue-spreading nose piece 76, which is attached to the distal exterior of the middle conduit 54.

The internal slope of the annular orifice 76 makes the removed wall core 46 smaller than the inside of inner conduit 58 to keep it from getting plugged. Dimples 78 on the distal inner side of the middle conduit 54 keep the inner conduit 58 centered in the nose piece, thereby keeping the annular orifice 76 annularly uniform. The area of the orifice 76 is adjustably controlled by adjusting the longitudinal location of the longitudinally displaceable inner conduit 58 and then fixing it in place. An annular protuberance 79 of the nose piece 74, as shown in FIG. 3, forms a seal with the main channel 27 in the heart wall 30, to prevent the leakage of spent tributary fluid from entering the bloodstream in the ventricle of the heart. It uses the flexibility of the heart, rather than an elastomer, to form the seal. It is made large enough to form a seal but not so large as to create trauma.

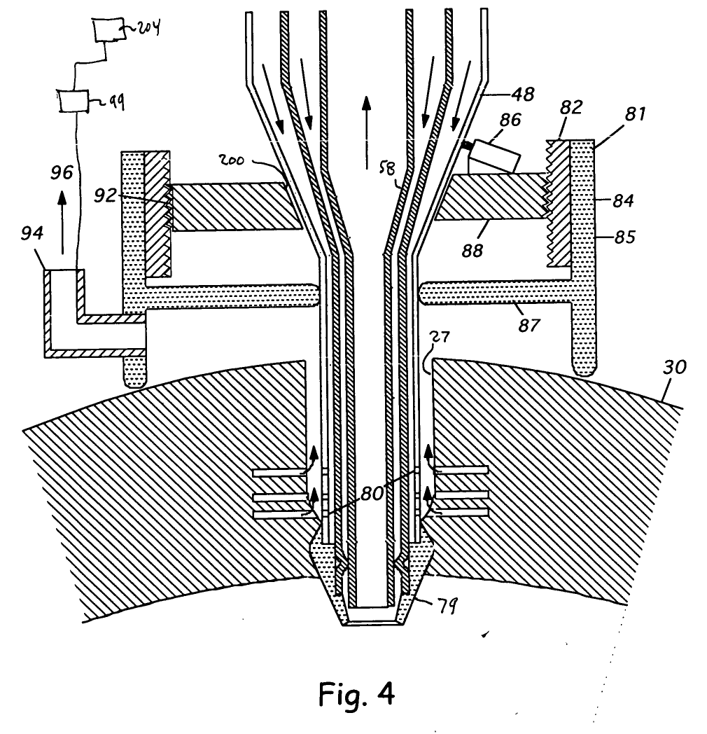

A collector ring 81, as shown in FIG. 4, comprises an elastomer seal 84 attached to a metal threaded ring 82. The elastomer seal 84 comprises an annular elastomer sleeve 85 attached to an annular elastomer washer 87. The washer 87 is flexible enough to allow the nose piece protrusion 79 to pass through its central opening during the introduction and removal of the nozzle 48 therethrough. A microswitch 86 is arranged in communication with the computer control 204 through a proper circuit 89, to control advance and pressure of the nozzle arrangement 48 during a treatment procedure. A vacuum source 99 is shown in fluid communication with the connector 94 and is also in controlled communication with the controller unit 204 for vacuum/pressure conformance.

At the start of revascularization procedure, the annular distal edge of the collector ring 81 is pressed against the outer surface of the wall 30 of the heart being treated. The pressure of the ring 81 causes the lower edge of sleeve 85 to seal against the surface of the heart, while the inner annular opening of washer 87 seals against the nozzle assembly 48. This enables a suction to be maintained in the plenum defined by he ring 81 and the surface of the heart, for removal of cellular debris and water-jet flow 96 out a side channel fitting 94 to a suction hose, not shown for clarity.

The conical hole 200 of the longitudinally adjustable, threaded disk 88 mates with the distalmost end of the conical surface of the expander 6 shown in FIG. 1, to control and limit the heartward advancing motion of the nozzle 48 therein. Without this depth penetration control, the nose piece protrusion 79 might extend beyond the end of the main channel 27, eliminating its sealing during formation of the tributary channels 72. The position of disk 88 is adjusted with the use of threads 92 by rotating the disk 88 in the ring 81.

Pressurized fluid-jet fluid is supplied by a controlled pressure supply system 202 represented in FIG. 5. It includes a reservoir 130 for containing fluid-jet fluid 132. The fluid may be heated by a heater 134 to a temperature controlled by a thermometer 136. The lid 138 of the reservoir includes a vent 140 and a fluid intake port 142. The fluid-jet fluid 144 is controllably withdrawn through conduit 146 by the suction of pump 148 and compressed, leaving through conduit 154.

When a main channel is being formed, solenoid valve 156 is opened, allowing fluid-jet fluid to flow through conduit 158 to nozzle 2. At least a portion of conduit 158 is a hose in communication with the nozzle assembly 48.

The controlled flow from the pump is determined by the controlled opening of for example, a pair of solenoid valves #1 and #2, both of which are normally closed. In order to avoid having to cycle the motor for pump 148 and prevent over-pressuring the system 202 when both solenoid valves are closed, the fluid-jet fluid 150 is recycled to the reservoir 130 through a return conduit 172, a back pressure regulator 174, and a conduit 152. The regulator 176 is adjusted to a sufficiently high pressure to remain shut when either of the solenoid valves is open. The system 202 is controlled and timed through a computer controller 204 in proper communication with the system 202 and sensors, arranged within the handle 10 and nozzle arrangements 20 and 48, for control of vacuum removal of debris, and for timing and force/pressure sensing of the pressurized fluid in the nozzle assemblies 20 and 48, the control system not being fully shown for clarity of the drawings.

We claim:

1. A surgical procedure for revascularization the myocardium, comprising:
directing a generally cylindrically shaped first nozzle into a heart wall being treated;
forming a first or main channel in said heart wall from the ventrical into the myocardium of the heart by said nozzle;
removing a generally cylindrically shaped tissue core through said nozzle, from said heart wall during formation of said first or main channel.

2. The surgical procedure as recited in claim 1, including:
placing a heart wall tissue cutting arrangement on a distal end of said nozzle; and
placing a heart wall tissue withdrawing arrangement on a proximal end of said nozzle.

3. The surgical procedure as recited in claim 2, including:
supporting a first conduit co-axially around said first nozzle so as to form a longitudinally directed first annular passageway between said nozzle and said first conduit;
providing a pressurized cutting fluid into said first passageway from a controllable pressurized fluid source to direct pressurized fluid from a distal end of said nozzle, for cutting said heart wall.

4. The surgical procedure as recited in claim 2, wherein said tissue cutting arrangement comprises a sharpened annular edge on said distal end of said nozzle.

5. The surgical procedure as recited in claim 2, wherein said tissue withdrawal arrangement comprises a vacuum conduit in communication with said first nozzle to facilitate removal of said tissue core from said heart wall and through said first nozzle.

6. The surgical procedure as recited in claim 3, including:
forming a chamfered distal end on said nozzle and said first conduit so as to direct said cutting fluid in an inward conically shaped direction to reduce the diameter of a tissue core removed from said wall.

7. The surgical procedure as recited in claim 3, including:
arranging a plurality of sideway directed orifices through said first conduit adjacent said distal end thereof, and
jetting a pressurized fluid through said sideway directed orifices to generate a plurality of tributary channels in said main channel in said wall of the heart being treated.

8. The surgical procedure as recited in claim 3, including:
inserting a second conduit between said nozzle and said first conduit to define a further annular passageway;
introducing a pressurized fluid into both said first annular passageway and said second passageway from a pressurized fluid source, to provide tissue cutting of said heart wall and tp provide tissue debris removal means therewith.

9. The surgical procedure as recited in claim 8,
arranging a tissue engaging nose piece arrangement on the distal end of said second conduit so as to provide a tissue sealing arrangement adjacent an annular pressurized fluid-emitting orifice thereat.

10. The surgical procedure as recited in claim 9, including:
moving said nozzle longitudinally so as to dimensionally alter said annular pressurized fluid emitting orifice.

11. The surgical procedure as recited in claim 3, including;
placing a longitudinally adjustable heart wall engaging plenum adjacent said distal end of said nozzle;
attaching said plenum into communication with a vacuum source to remove debris from said heart wall during a channel generating revascularization procedure.

12. The surgical procedure as recited in claim 11, including:
forming a main channel into a heart wall;
forming a plurality of tributaries generally perpendicular with respect to said main channel and into said wall of the heart; and
evacuating debris from said tributaries through an outer annular channel while evacuating core tissue from said main channel through said nozzle.

13. An apparatus for performing a revascularization procedure on a myocardium, comprising:
an elongated hollow nozzle having a proximal end and a tissue piercing distal end; and
a first conduit arranged co-axial with said nozzle to define an annular fluid directing passageway therebetween, and a controllable pressure source in communication with said passageway.

14. The apparatus as recited in claim 13, including:
a vacuum arranged in communication with said proximal end of said nozzle to withdraw a core plug of tissue from said nozzle.

15. The apparatus as recited in claim 13, including:
an annular plenum for sealing said nozzle during a procedure and for withdrawing debris generated therewith.

16. The apparatus as recited in claim 15, wherein said plenum has an adjustment means thereon to limit the depth of a nozzle may travel into a heart wall.
